(19) United States
(12) Patent Application Publication
(10) Pub. No.: US 2025/0367010 A1
(43) Pub. Date: Dec. 4, 2025

(54) VARIABLE DIAMETER STENTS

(71) Applicant: Starlight Cardiovascular, Inc., San Diego, CA (US)

(72) Inventors: Mark Juravic, Encinitas, CA (US); Thomas Duerig, Fremont, CA (US)

(21) Appl. No.: 18/875,670

(22) PCT Filed: Jun. 15, 2023
(86) PCT No.: PCT/US23/68516

§ 371 Date: Dec. 16, 2024

Related U.S. Application Data
(60) Provisional application No. 63/352,952, filed on Jun. 16, 2022.

(51) Int. Cl. A61F 2/915

(52) U.S. Cl. CPC A61F 2/915; A61F 2002/91583; A61F 2250/001; A61F 2250/0048

(57) ABSTRACT

Described herein are variable diameter stents for insertion into a blood vessel lumen to provide a generally radially outward force on the blood vessel lumen. The stent may comprise a first end section comprising a first plurality of struts defining a first variable diameter; a second end section comprising a second plurality of struts defining a second variable diameter; a body section extending between the first end section and the second end section and comprising a third plurality of struts; and a device lumen extending through the first end section, the body section, and the second end section. In an expanded configuration, a first subset of the first plurality of struts is increased in length from the body section towards a proximal face, and a first subset of the second plurality of struts is increased in length from the body section towards a distal face.

### CROSS-REFERENCE TO RELATED APPLICATIONS

This application claims the priority benefit of U.S. Provisional Application No. 63/352,952, filed on Jun. 16, 2022, the disclosure of which is herein incorporated by reference in its entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates generally to the field of intraluminal devices, and more specifically to the field of stenting.

### BACKGROUND

Stents are used in a wide variety of coronary, neurovascular, and peripheral vasculature procedures. While stents have been used for several decades, many stents still present challenges, including conformability, apposition to vessel walls, and fixing. Further, some technical challenges faced by pediatric cardiovascular physicians (surgeons and interventionalists alike) have long been ignored, forcing them to use devices designed for adults and different conditions to treat ailing babies with very specific anatomical considerations. One such case is in the sustained opening of the ductus arteriosus, a natural conduit that exists in all newborns but closes shortly after birth. In certain congenital heart defects, it is crucial to maintain ductus patency for the newborn to survive without surgical intervention.

### SUMMARY

In general, there exists a need for new devices and methods for maintaining a patent ductus arteriosus. In some aspects, the techniques described herein relate to a device for insertion into a blood vessel lumen to provide a generally radially outward force on the blood vessel lumen, including: a first end section including a first plurality of struts defining a first variable diameter and a proximal face; a second end section including a second plurality of struts defining a second variable diameter and a distal face; a substantially cylindrical body section extending between the first end section and the second end section and including a third plurality of struts; and a device lumen extending through the first end section, the substantially cylindrical body section, and the second end section, the device lumen being configured to allow blood flow through the device, wherein, in an expanded configuration, the first variable diameter of the first end section increases from the substantially cylindrical body section towards the proximal face, and the second variable diameter of the second end section increases from the substantially cylindrical body section towards the distal face, and wherein a first subset of the first plurality of struts is increased in length from the substantially cylindrical body section towards the proximal face, and a first subset of the second plurality of struts is increased in length from the substantially cylindrical body section towards the distal face.

In some aspects, the techniques described herein relate to a device, wherein the device is configured for a treatment of ductus arteriosus. In some aspects, the techniques described herein relate to a device, wherein: the body section including the third plurality of struts is configured to have a first radial force at a first end of the body section and at a second end of the body section, the first end being opposite the second end; the first plurality of struts of the first end section are arranged to have a second radial force that substantially matches the first radial force; and the second plurality of struts of the second end section are arranged to have a third radial force that substantially matches the first radial force.

In some aspects, the techniques described herein relate to a device, wherein the first radial force is between about 0.4 N/mm to about 0.5 N/mm when the device is at about 2 mm of compression. In some aspects, the techniques described herein relate to a device, wherein the first radial force is between about 0.2 N/mm to about 0.6 N/mm when the device is at about 2 mm of compression. In some aspects, the techniques described herein relate to a device, wherein the length of the first subset of the first plurality of struts is increased by about 5 percent to about 25 percent moving from the substantially cylindrical body section towards the proximal face.

In some aspects, the techniques described herein relate to a device, wherein the length of the first subset of the second plurality of struts is increased by about 12 percent to about 25 percent moving from the substantially cylindrical body section towards the distal face. In some aspects, the techniques described herein relate to a device, wherein each of the third plurality of struts is substantially equal in length. In some aspects, the techniques described herein relate to a device, wherein: the first subset of the first plurality of struts have a length of about 1 mm to about 2 mm; and the first subset of the second plurality of struts have a length of about 1 mm to about 2.1 mm.

In some aspects, the techniques described herein relate to a device, further including a second subset of struts in the first plurality of struts and a second subset of struts in the second plurality of struts, wherein: the second subset of struts in the first plurality of struts have a length of about 1.2 mm to about 2.5 mm; and the second subset of struts in the second plurality of struts have a length of about 1.4 mm to about 2.5 mm.

In some aspects, the techniques described herein relate to a device, further including a third subset of struts in the first plurality of struts and a third subset of struts in the second plurality of struts, wherein: the third subset of struts in the first plurality of struts have a length of about 1.9 mm to about 2.0 mm; and the third subset of struts in the second plurality of struts have a length of about 1.9 mm to about 2.0 mm. In some aspects, the techniques described herein relate to a device, wherein the first variable diameter of the first end section is increased from the substantially cylindrical body section towards the proximal face by about 40 percent to about 80 percent.

In some aspects, the techniques described herein relate to a device, wherein the first end section has a flare shape that flares from the body section toward the proximal face. In some aspects, the techniques described herein relate to a device, wherein the second variable diameter of the second end section is increased from the substantially cylindrical body section towards the distal face by about 40 percent to about 80 percent. In some aspects, the techniques described herein relate to a device, wherein the second end section has a flare shape that flares from the body section toward the distal face.

In some aspects, the techniques described herein relate to a device, wherein the first plurality of struts and the second plurality of struts are arranged in rings. In some aspects, the techniques described herein relate to a device, wherein there are between about 3 rings to about 5 rings including each of the first end section and the second end section.

In some aspects, the techniques described herein relate to a device, wherein the increase in first and second variable diameters moving from the substantially cylindrical body section either proximally or distally, respectively, is incremental on a ring-by-ring basis. In some aspects, the techniques described herein relate to a device, wherein the increment is an increase in the first and second variable diameters of between about 10 percent to about 30 percent.

In some aspects, the techniques described herein relate to a device, wherein, in the expanded configuration, adjacent struts in each ring are arranged to provide an outward radial resistive force throughout the first end section and the second end section, wherein the outward radial resistive force ranges from about 0.1 N/mm to about 0.4 N/mm at 1 mm of compression and about 0.1 N/mm to about 0.6 N/mm at 2 mm of compression.

In some aspects, the techniques described herein relate to a device, wherein each ring is connected to an adjacent ring by about 3 bridges to about 9 bridges. In some aspects, the techniques described herein relate to a device, wherein the bridges are positioned such that crowns in adjacent rings are substantially aligned.

In some aspects, the techniques described herein relate to a device, wherein one or both of: the proximal face and the distal face has a diameter that is about 10 percent to about 80 percent larger than a diameter of the substantially cylindrical body section.

In some aspects, the techniques described herein relate to a device for insertion into a blood vessel lumen to provide a generally radially outward force on the blood vessel lumen, including: a first end section including a first plurality of rings each including a first plurality of struts, wherein the first end section defines a first variable diameter and a proximal face; a second end section including a second plurality of rings each including a second plurality of struts, wherein the second end section defines a second variable diameter and a distal face; a substantially cylindrical body section extending between the first end section and the second end section and including a third plurality of struts; and a device lumen extending through the first end section, the substantially cylindrical body section, and the second end section, the device lumen being configured to allow blood flow through the device, wherein, in an expanded configuration, each ring of the first plurality of rings in the first end section has an increased first diameter moving from the substantially cylindrical body section towards the proximal face, and each ring of the second plurality of rings in the second end section has an increased second diameter moving from the substantially cylindrical body section towards the distal face, and wherein, in an expanded configuration, the first plurality of struts of each ring of the first plurality of rings has an increased first length moving from the substantially cylindrical body section towards the proximal face, and the second plurality of struts of each ring of the second plurality of rings has an increased second length moving from the substantially cylindrical body section towards the distal face.

In some aspects, the techniques described herein relate to a device, wherein the device is configured to be positioned in a bodily lumen including a procedure diameter that is larger than a post-procedure diameter, and wherein the first and second end sections are configured to anchor the device in the bodily lumen having the procedure diameter. In some aspects, the techniques described herein relate to a device, wherein the procedure diameter of the bodily lumen is a result of prostaglandin administration to a patient.

In some aspects, the techniques described herein relate to a device, wherein: the body section including the third plurality of struts is configured to have a first radial force at a first end of the body section and at a second end of the body section, the first end being opposite the second end; the first plurality of struts of each ring of the first plurality of rings are arranged to have a second radial force that substantially matches the first radial force; and the second plurality of struts of each ring of the second plurality of rings are arranged to have a third radial force that substantially matches the first radial force.

In some aspects, the techniques described herein relate to a device, wherein the first radial force is between about 0.1 N/mm to about 0.6 N/mm when the device is at about 2 mm of compression.

In some aspects, the techniques described herein relate to a device for insertion into a blood vessel lumen to provide a generally radially outward force on the blood vessel lumen, including: a first end section including a first plurality of struts defining a first variable diameter and a proximal face; a second end section including a second plurality of struts defining a second variable diameter and a distal face; a substantially cylindrical body section extending between the first end section and the second end section and including a third plurality of struts; and a device lumen extending through the first end section, the substantially cylindrical body section, and the second end section, the device lumen being configured to allow blood flow through the device, wherein, in an expanded configuration, the first variable diameter of the first end section increases from the substantially cylindrical body section towards the proximal face, and the second variable diameter of the second end section increases from the substantially cylindrical body section towards the distal face, and wherein a first subset of the first plurality of struts is increased in length from the substantially cylindrical body section towards the proximal such that a relatively constant crown angle is maintained throughout the body section of the device, and a second subset of the second plurality of struts is increased in length from the substantially body section towards the distal face such that a relatively constant crown angle is maintained throughout the body section of the device.

The illustrated embodiments are merely examples and are not intended to limit the disclosure. The schematics are drawn to illustrate features and concepts and are not necessarily drawn to scale.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIGS. 1A-1C show one example embodiment of a stent.

FIG. 2 shows another example embodiment of a stent.

FIG. 3 shows another yet another example embodiment of a stent.

FIG. 4 shows another example embodiment of a stent.

FIG. 5 shows another example embodiment of a stent.

FIG. 6 shows a stent cut pattern with struts having variable length, moving from a body section of the stent to a first or second end section.

FIG. 7 shows a zoomed-in view of crown angles between adjacent struts in each ring.

FIG. 8 shows example ring diameter of a first or second end section of a stent in an expanded configuration.

FIG. 9 shows crown alignment between adjacent rings when the stent is in an expanded configuration.

FIG. 10 shows a graphical representation of example radial forces versus diameter for the stents described herein.

FIG. 11 shows a flow diagram of an example method of treatment of ductus arteriosus using at least one of the stents described herein.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated systems, methods, and/or devices described herein. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

Described herein are various stent embodiments and methods for delivering such stents. The stents described herein may include one or more varied strut lengths that cause a change in one or more crown angles of the stent to ensure that the stent, expanded to a particular diameter, maintains a substantially equal radial force along a longitudinal length of the stent. In addition, the stent embodiments described herein may have a stent diameter that varies along the longitudinal length while maintaining a relatively constant radial force along the longitudinal length when the stent is positioned in a lumen that is oversized relative to the stent diameter. For example, the stents described herein may have a first stent diameter in a center portion while end portions may progressively increase in diameter from the center portion to each respective end portion.

In general, the stents described herein provide an improved way of maintaining patency for vessels experiencing significant changes between a systolic diameter and a diastolic diameter, significant forces from flow patterns that may cause the stent to migrate, changes in anatomy (e.g., a ductus arteriosus attempting to close), or other situations causing significant forces or diameter changes. For example, because strut length may progressively increase when moving from a center portion of the stent to an end portion, crown angles may progressively increase to maintain a predefined radial force along the length of the stent. The progressive increase in strut length and corresponding increase in crown angle may ensure that the stent is stable with a radial force that ensures that the stent maintains patency in the vessel.

In some embodiments, the stents described herein are for treating and/or managing patient conditions associated with a ductus arteriosus. These embodiments may be designed to address the challenges facing treating physicians; including a right-sized delivery system; an end-to-end annular coverage of the ductus arteriosus; insertion; navigation, and deployment through tortuous ductus anatomy; and precise placement to avoid stent protrusion into the aorta and/or pulmonary arteries, which can also ensure avoidance of additional surgeries to adjust and correct the placement of the stent. Embodiments of the stents and their method of delivery and placement that are designed and tested specifically for this purpose will decrease reinterventions, morbidity, vasospasm, and potentially mortality for patients with ductal-dependent circulation.

Approximately 2,000 babies are born in the United States yearly that could benefit from a ductus arteriosus stent, categorized into two groups: patients with ductal-dependent pulmonary circulation and patients with ductal-dependent systemic circulation.

Patients with ductal-dependent pulmonary circulation are conventionally treated with Modified Blalock-Taussig shunts (MBTS), a surgical procedure where the chest is opened, the neonate is put on cardiopulmonary bypass, and a plastic conduit is implanted to provide flow to the systemic and pulmonary circulations. MBTS carry a 7.2 percent risk of mortality and 13.1 percent risk of morbidity in the United States. Alternatively, conventional ductal stenting has resulted in similar or reduced mortality over MBTS and provides ductal-dependent pulmonary circulation while being minimally invasive. The conventional practice of stenting the ductus with “repurposed” coronary stents that are currently available carries a 47 percent rate of reintervention. Reintervention rates are higher when a portion of the stent extends into the pulmonary artery either partially or fully jailing one of the branch pulmonary arteries, which occurs in 21.9 percent of ductus stenting cases with “repurposed” coronary stents. A stent and delivery system designed and tested for maintaining ductus arteriosus patency could move patients from open surgery to a less invasive approach, with reduced mortality compared to MBTS and fewer reinterventions compared to conventional stenting with “repurposed” coronary stents.

Patients with ductal-dependent systemic circulation typically have Hypoplastic Left Heart Syndrome (HLHS). The first procedure in a three-stage palliation for HLHS is typically performed in the first two weeks of life, and a hybrid procedure which uses ductal stenting could prevent cardiopulmonary bypass based procedures in these neonates. The ductus arteriosus stents described herein could also address the HLHS patient population by modifying the stent diameter and addressing aortic impingement.

The conventional coronary stents that are repurposed for ductal stenting are all balloon-expandable, resulting in some limitations in radial force and foreshortening with balloon deployment. Balloon-expandable systems can also be relatively stiff on the distal end with the crimped stent loaded over the balloon material, making tracking through tortuous anatomy challenging. Also unsuccessfully, previously designed self-expanding stents that have sufficient flexibility to advance through the tortuous anatomy, while loaded in the delivery system, have been shown to have insufficient radial force or kink resistance to maintain an open lumen.

Furthermore, issues that may arise when using conventional repurposed stents in the ductus arteriosus include: 1) a lack of understanding of ductus tissue-stent interaction for selecting a stent with the proper radial force; 2) challenging measurement of the 3D ductus arteriosus with 2D angiography, making stent sizing difficult; 3) mechanical properties of the stent and delivery system change the ductus tortuosity and length, further complicating stent sizing (e.g., the stent may straighten the ductus or elongate the ductus); 4) difficulty in precise stent placement to prevent protrusion into surrounding arteries; 5) delivery systems are designed for adult vessels, risking damage to the smaller, vulnerable blood vessels from percutaneous access to placement location; 6) conventional delivery systems are not designed for the approach angles or deployment in tortuous ductus anatomies; and (7) at least for pulmonary dependent circulation, with balloon stents, a practitioner has to preselect the size of the stent for the ductus, which can be problematic if the selection is a mismatch with particular patient anatomy. For example, the stent cannot be too big (e.g., too large of an inner or outer diameter) because an oversized stent can cause an overage of blood flow to lungs. In such a setting, the practitioner cannot use prostaglandins during the procedure to get the ductus to the desired size (i.e., the practitioner cannot estimate the size that the ductus should be if the ductus is dilated on prostaglandins). Further, the conventional approach of removing prostaglandins prior to the procedure has a high risk of vasospasm, which can be dangerous and/or life threatening for the patient.

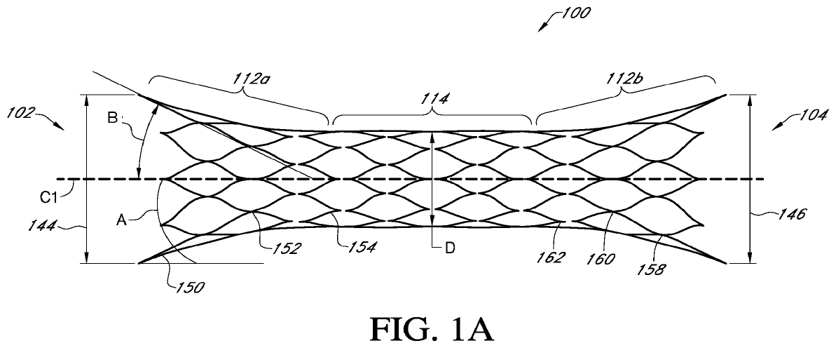

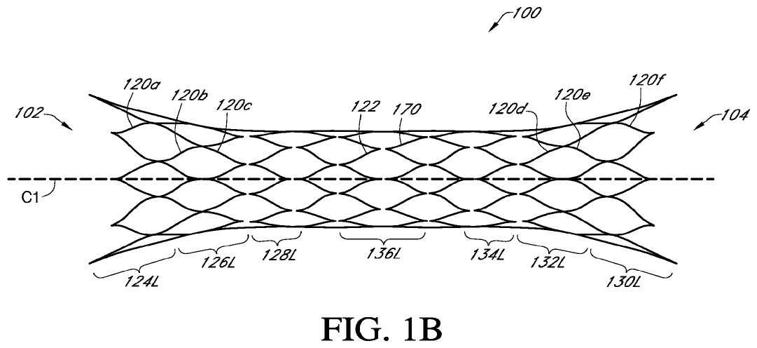

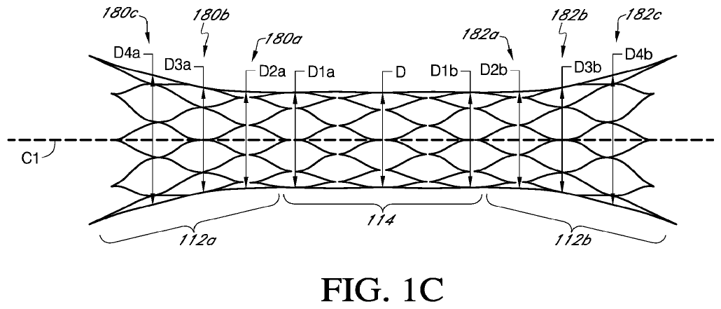

The stent embodiments described herein solve the above technical problems with technical solutions. For example, the stents described herein may have an optimized and/or relatively constant radial force along a longitudinal length (along centerline axis C1 in FIG. 1A) to maintain ductus arteriosus patency. The features that may ensure such a radial force include strut length variations, crown angle variations, and/or stent material variations. Such features may address operational use and stent placement challenges described above. The stents described herein ensure accurate delivery and placement as well as annular coverage over a length of a patient's ductus arteriosus, to maintain patency of the ductus, without interference with, or substantial protrusion into, the adjacent aorta or pulmonary artery.

Further, any of the stent embodiments described herein may be delivered with a catheter or a microcatheter. Microcatheters offer distinct advantages for maintaining a patent ductus arteriosus. For example, microcatheters are more deliverable to access difficult anatomy compared to balloon mounted stents, and microcatheters enable smaller access sites.

In any of the embodiments or figures shown and/or described herein, a catheter or microcatheter may be used as part of a delivery system. Selection of a catheter or microcatheter may be used herein depending on physician preference, type of circulation desired (e.g., pulmonary vs. systemic), size of stent, size of the ductus arteriosus of the patient, etc.

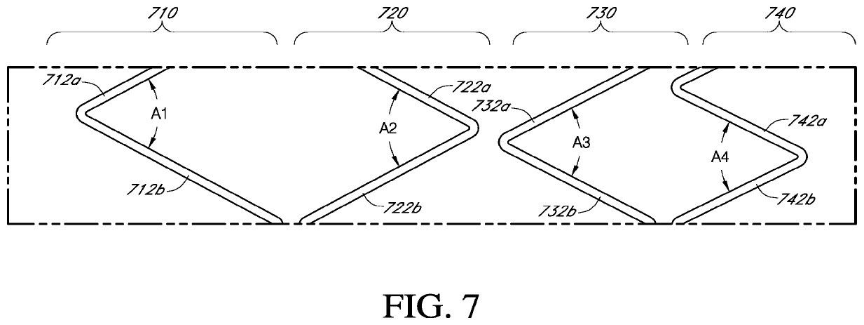

As used herein, the term “crown angle” refers to an angular measurement derived based on the lengths of two adjacent struts forming an angle at a crown portion of a stent. Angles A1, A2, A3, and A4 of FIG. 7, for example, may represent crown angles within a stent.

Advantageously, embodiments of stents (e.g., ductus arteriosus stents) described herein may be positioned to precisely cover an annular region of the ductus arteriosus with a single stent to maintain patency while not inhibiting blood flow through adjacent arteries. The stents described herein (e.g., FIGS. 1A-9) can be positioned along the length of the ductus and also enable changes of the stent length during stent delivery and placement to ensure coverage of an entire length of the ductus. Any of the stent embodiments described herein may also support the tissue of the ductus along its length to prevent closure of the ductus.

In general, the stents described herein include a first end section, a body section, and a second end section. In some embodiments, the first end section and/or the second end section is flared, such that the first end section and/or the second end section is oversized relative to the body section to improve stent fixing.

In general, the stents described herein may be configured for use in vessels having a larger inner diameter, such that the outer diameter of the first and/or second end sections of the stent may be oversized relative to the vessel inner diameter and an outer diameter of a body section of the stent may be undersized. This stent structure may function to set the minimum diameter of the stent in order to control the amount of flow through the stent.

In some embodiments, the stents described herein may be used in a method associated with or providing treatment of ductus arteriosus in a pediatric patient. For example, the stents described herein may be used in a method of maintaining a patent ductus arteriosus in a pediatric patient to increase a pulmonary circulation of the pediatric patient for a time period. In some embodiments, the method includes deploying a first end of a self-expanding stent at a first end of a lumen defined by a ductus arteriosus of a subject, anchoring at least a portion of a first flange of the first end of the stent such that the first flange at least partially circumferentially covers one of: a pulmonary artery ostium or an aortic ostium, deploying a second end of the stent, such that a stent body covers an entire length of the lumen defined by the ductus arteriosus, and anchoring at least a portion of a second flange of the second end of the stent such that the second flange at least partially circumferentially covers the other of the pulmonary artery ostium or the aortic ostium. The anchoring of the at least one portion of the first flange and the anchoring of the at least one portion of the second flange functions to maintain a patent ductus arteriosus for the subject.

In some embodiments, a bodily lumen may have a larger diameter during a procedure or pre-procedure than after a procedure. For example, in ductus arteriosus cases, a patient may be on prostaglandin therapy resulting in the ductus arteriosus having a larger diameter during stenting and then a smaller diameter once the stent is placed and prostaglandin therapy is removed. In other embodiments, the bodily lumen may be expanded using a balloon or similar mechanism during stent positioning and then the balloon removed after the stent is positioned. In such embodiments, one or both of the first and second end sections function (by way of their variable diameter) to anchor the stent in the bodily lumen while the diameter of the bodily lumen is increased.

In general, and as described herein, a number of struts per ring, a length of each strut, and a final expansion diameter of the stent will dictate an angle between adjacent struts in the same ring. If a crown angle is too large (e.g., because the strut lengths are too short), the stent cannot be expanded to its desired diameter. If a crown angle is too small (e.g., because the strut lengths are too long), then the stent may not maximize its radial force potential. The stents described herein may incorporate increasing strut lengths as the outer diameter of the stent increases, such that a relatively constant crown angle and/or relatively constant radial force is maintained throughout the body of the stent. In some embodiments, the stents described herein alternatively, may incorporate increasing strut lengths as the outer diameter of the stent increases, such that a crown angle increases as the diameter increases.

Various stent embodiments shown and described herein include oversized or flared first and/or second end sections. In some embodiments, an outer diameter of the stent may increase from a body section to a first and/or second end section. In some embodiments, a body section of the stent may have a substantially constant diameter while the first and/or second end sections increase in diameter moving outward from the body section to the proximal or distal faces, respectively, of the stent. As such, the first and/or second end sections have a variable diameter and/or variable strut length. For example, a terminal ring may have the largest diameter; a diameter of the penultimate ring may be smaller than the diameter of the terminal ring; and a diameter of the antepenultimate ring may be smaller than the diameter of the penultimate ring. In embodiments including more than three rings, a diameter of the fourth ring may be smaller than the diameter of the antepenultimate ring.

Any of the stents described herein may transition from a crimped configuration to an expanded configuration. For pediatric use cases, for example in the heart, a crimped diameter of the device is less than about 0.8 mm and an expansion diameter of the device is greater than about 3 mm measured at the body section. For coronary applications, a crimped diameter of the device may be less than about 1.78 mm (to fit within a 5F introducer) and an expansion diameter may be about 2.5 mm to about 4.5 mm measured at the body section of the stent. For neuroanatomy applications, an expansion diameter may be about 2.5 mm to 4 mm measured at the body section. For peripheral applications, a crimped diameter may be less than about 2.03 mm (to fit within a 6F introducer) and an expansion diameter may be about 5 mm to about 10 mm measured at the body section.

Any of the stents described herein, in an expanded configuration, may have a radial resistive force (based on ISO 25539 standards), at about 2 mm of compression, of about 0.4 N/mm to about 0.5 N/mm. In another example, when the stents described herein are in an expanded configuration, a radial resistive force (based on ISO 25539 standards), at about 1 mm of compression, may be greater than about 0.10 N/mm; about 0.10 N/mm to about 0.4 N/mm; about 0.2 N/mm to about 0.3 N/mm; about 0.3 N/mm to about 0.4 N/mm; or about 0.35 N/mm to about 0.4 N/mm. For example, when the stent is compressed from a diameter of about 4 mm to about 3 mm, the radial resistive force may be about 0.25 N/mm to about 0.27 N/mm. In another example, when the stent is compressed from a diameter of about 4 mm to about 2 mm, the radial resistive force may be about 0.1 N/mm to about 0.6 N/mm; about 0.1 N/mm to about 0.2 N/mm; about 0.2 N/mm to about 0.3 N/mm; about 0.3 N/mm to about 0.4 N/mm; about 0.4 N/mm to about 0.5 N/mm; or about 0.5 N/mm to about 0.6 N/mm. Such radial resistive force parameters may be applicable for pediatric use cases, for example for maintaining a patent ductus arteriosus or a patent septal conduit. For peripheral applications (e.g., femoral or iliac vessel stenting), a radial resistive force may be about 0.4 N/mm to about 2 N/mm; about 0.4 N/mm to about 0.7 N/mm; or about 1 N/mm to about 1.75 N/mm. For coronary applications, a radial resistive force may be about 0.8 N/mm to about 2 N/mm; about 1 N/mm to about 1.75 N/mm; or about 0.8 N/mm to about 1.4 N/mm.

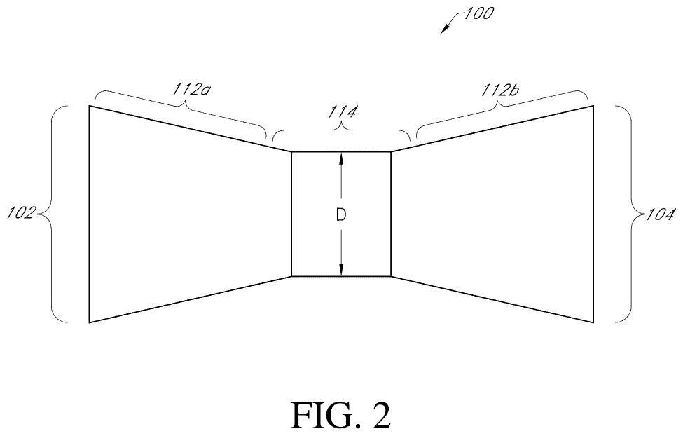

FIGS. 1A-1C and FIG. 2 show one embodiment of a stent having a variable diameter. As shown, the stent 100 has a first end section 112a defining a proximal face 102, a second end section 112b defining distal face 104, and a body section 114 between the first end section 112a and the second end section 112b. As shown in FIG. 1A, proximal face 102 of the first end section 112a has a diameter 144 (measured at the terminal crowns of the terminal ring) that is about 20 percent to about 50 percent or about 20 percent to about 30 percent larger than a diameter D of the body section 114. First end section 112a includes at least one ring, one or more rings, or a plurality of rings.

As shown in FIG. 1A, the first end section 112a includes a terminal ring 150, a penultimate ring 152, and an antepenultimate ring 154. The end section 112a may extend radially away from a centerline axis C1 by an angle A (measured between centerline axis C1 and an end of terminal ring 150) and/or angle B (measured between centerline axis C1 and an end of terminal ring 150). Similarly, the end section 112b may extend radially away from a centerline axis C1 by the angle A (measured between centerline axis C1 and an end of terminal ring 158) and/or angle B in a similar fashion. The angle A may be between about 40 degrees to about 90 degrees; about 45 degrees to about 85 degrees; about 50 degrees to about 80 degrees; or about 55 degrees to about 75 degrees; or substantially about 65 degrees. The angle B may be between about 40 degrees to about 90 degrees; about 45 degrees to about 85 degrees; about 50 degrees to about 80 degrees; or about 55 degrees to about 75 degrees; or substantially about 65 degrees.

In some embodiments, the stent 100 (or any of the stents described herein) may incorporate increasing strut lengths as the outer diameter of the stent increases to maintain stability in the stent, while increasing the crown angles as the outer diameter of the stent increases in order to increase (and/or maintain) the radial force of each ring of the stent. The crown angle may increase from a body section 114 toward the first end section 112a and/or from a body section 114 toward the second end section 112b. The crown angle may increase by about 1 percent to about 10 percent; about 2 percent to about 8 percent; about 3 percent to about 5 percent; about 4 percent to about 5 percent from the body section 114 toward either or both of the first end section 112a and the second end section 112b. In some embodiments, the stent 100 (or any of the stents described herein) may include crown angles that are held substantially constant as the stent diameter increases.

Rings 150 and 152 and rings 152 and 154 are connected to each other via one or more or a plurality of bridges. Each bridge has a length of about 0.1 mm to about 0.25 mm. There may be about three bridges to about nine bridges. Terminal ring 150 includes a plurality of struts 120a, each having length 124L; penultimate ring 152 includes a plurality of struts 120b, each having length 126L; and antepenultimate ring 154 includes a plurality of struts 120c, each having length 128L. Length 124L of each strut 120a may be substantially similar to length 126L of each strut 120b and/or length 128L of each strut 120c. Preferably, length 124L is longer than length 126L, which is longer than length 128L, such that the lengths of the struts increase moving from the body section 114 to the first end section 112a to the proximal face 102. In other embodiments, length 128L is longer than length 126L, which is longer than length 124L, such that the lengths of the struts decrease moving from the body section 114 to the first end section 112a to the proximal face 102. In a further iteration, length 128L and 126L may be substantially the same or 128L and 124L may be substantially the same or length 126L and 124L may be substantially the same. Strut length 124L, 126L, and 128L may each be between about 2.5 mm and about 4.5 mm. Preferably, a length 124L of each strut 120a may be about 1.8 mm to about 2.3 mm; about 1.8 mm to about 2.0 mm; about 1.9 mm to about 2.0 mm. The length 126L of each strut 120b may be about 1.6 mm to about 2.0 mm; about 1.6 mm to about 1.8 mm; or about 1.7 mm to about 1.8 mm. The length 128L of each strut 120c may be about 1.3 mm to about 1.7 mm; about 1.3 mm to about 1.6 mm; about 1.4 mm to about 1.6 mm; or about 1.5 mm to about 1.6 mm.

Various stent embodiments shown and described herein include oversized or flared (e.g., having a flared shape) first and/or second end sections. For example, a first end section (e.g., section 112a of FIG. 1B) may flare from a first end portion of a body portion of the stent 100 (e.g., the substantially cylindrical body section 114 of FIG. 1B) toward the proximal face 102 of the stent 100. Similarly, a second end section (e.g., section 112b of FIG. 1B) may flare from a second end portion (opposite the first end portion) of the body portion of the stent 100 toward the distal face 104 of the stent 100.

Referring to FIG. 1C, the stent 100 may be further sectioned into subsets of struts. For example, a number of struts 120c (FIG. 1B) may be included in a first subset 180a of the first end section 112a. Each strut 120c in the first subset 180a of the first end section 112a may have a length of about 1.5 mm to about 1.6 mm. Similarly, the stent 100 may include a number of struts 120d that may be included in a first subset 182a of the second end section 112b. Each strut 120d in the first subset 182a of the second end section 112b may have a length of about 1.5 mm to about 1.6 mm.

The stent 100 may further include a second subset 180b of struts 120b in the first end section 112a. Each strut 120b in the second subset 180b of struts in the first end section 112a may have a length of about 1.7 mm to about 1.8 mm. Similarly, the stent 100 may include a second subset 182b of struts 120e in the second end section 112b. Each strut in the second subset 182b of struts 120e in the second end section 112b may have a length of about 1.7 mm to about 1.8 mm.

The stent 100 may further include a third subset 180c of struts 120a in the first end section 112a. The third subset 180c of struts 120a in the first end section 112a may have a length of about 1.9 mm to about 2.0 mm. The stent 100 may further include a third subset 182c of struts 120f in the second end section 112b. Each strut in the third subset 182c of struts 120f in the second end section 112b may have a length of about 1.9 mm to about 2.0 mm.

In a non-limiting example embodiment, the strut length of the body section 114 may have a strut length of about 1.53 mm. Each strut 120d and 120c in the respective first subsets 182a, 180a may have a length of about 1.55 mm. Each strut 120e, 120b in the respective second subsets 182b, 180b may have a length of about 1.77 mm. Each strut 120f, 120a in the respective third subsets 182c, 180c may have a length of about 1.93 mm.

As shown in FIGS. 1A-1B, distal face 104 of the second end section 112b has a diameter 146 (measured at the terminal crowns of the terminal ring) that is about 20 percent to about 75 percent; about 20 percent to about 30 percent; about 30 percent to about 40 percent; about 40 percent to about 50 percent; about 50 percent to about 60 percent; about 60 percent to about 70 percent; or about 70 percent to about 75 percent larger than a diameter D of the body section 114. Second end section 112b includes at least one ring, one or more rings, or a plurality of rings. As shown in FIG. 1A, the second end section 112b includes a terminal ring 158, a penultimate ring 160, and an antepenultimate ring 162. Rings 158 and 160 and rings 160 and 162 are connected to each other via one or more or a plurality of bridges. Each bridge has a length of about 0.1 mm to about 0.25 mm. There may be about three bridges to about nine bridges. Terminal ring 158 includes a plurality of struts 120f, each having length 130L; penultimate ring 160 includes a plurality of struts 120e, each having length 132L; and antepenultimate ring 162 includes a plurality of struts 120d, each having length 134L.

Length 130L of each strut 120f may be substantially similar to length 132L of each strut 120e and/or length 134L of each strut 120d. Preferably, length 130L is longer than length 132L, which is longer than length 134L, such that the lengths of the struts increase moving from the body section 114 to the second end section 112b to the distal face 104. In other embodiments, length 134L is longer than length 132L, which is longer than length 130L, such that the lengths of the struts decrease moving from the body section 114 to the second end section 112b to the distal face 104. In a further iteration, length 130L and 132L may be substantially the same and/or 130L and 134L may be substantially the same or length and/or 132L and 134L may be substantially the same. Strut length 130L, 132L, and 134L may each be between about 2.5 mm and about 4.5 mm. Preferably, a length 130L of each strut 120f may be about 1.2 mm to about 2.5 mm; about 1.2 mm to about 2.0 mm; or about 1.5 mm to about 2.0 mm; about 1.6 mm to about 2.1 mm; or about 1.9 mm to about 2.1 mm. The length 132L of each strut 120e may be about 1.6 mm to about 2.0 mm; about 1.6 mm to about 1.8 mm; or about 1.7 mm to about 1.8 mm. The length 134L of each strut 120d may be about 1.0 mm to about 2 mm; about 1.3 mm to about 1.6 mm; about 1.4 mm to about 1.8 mm; about 1.8 mm to about 1.9 mm; or about 1.9 mm to about 2.1 mm.

Body section 114 includes a plurality of rings 122, each including a plurality of struts 170. Body section 114 may include at least one ring, one or more rings, or a plurality of rings. For example, there may be about one ring, about 2 to about 6 rings, or about 3 to about 10 rings. The plurality of struts 170 of the body section 114 each have a length 136L.

As shown in FIG. 1B but also for any of the stent embodiments described herein, the length 136L of each of the struts 170 may be about 0.6 mm to about 1.6 mm, preferably about 1.0 mm to about 1.6 mm. The rings 122 of the body section 114 may be connected via a plurality of bridges, for example about three bridges to about nine bridges between each pair of adjacent rings. Each bridge has a length of about 0.1 mm to about 0.25 mm.

In some embodiments, the diameter across the stent 100 may vary. For example, FIG. 1C depicts a number of different diameters for stent 100. The diameter D represents the diameter for the body section 114. In some embodiments, a first end of the body section 114 has a diameter D1a that may be substantially equal to diameter D. Similarly, a second end of the body section 114 has a diameter D1b that may be substantially equal to diameter D. In some embodiments, diameter D1a and diameter D1b may be about zero to about 4 percent larger than diameter D; about 1 percent to about 4 percent larger than diameter D; or about 2 percent to about 3 percent larger than diameter D.

A diameter D2a and a diameter D2b may represent the diameter of the first subset 180a and the first subset 182a formed by struts of the respective first and second end sections 112a, 112b. The diameters D2a, D2b may be substantially larger than diameter D1a or D1b. In some embodiments, diameter D2a and diameter D2b may be about 2 percent to about 30 percent larger than diameter D1a or D1b; about 2 percent to about 8 percent larger than diameter D1a or D1b; or about 8 percent to about 15 percent larger than diameter D1a or D1b; or about 15 percent to about 30 percent larger than diameter D1a or D1b. In some embodiments, diameter D2a and diameter D2b may be about 5 percent larger than diameter D1a or D1b.

A diameter D3a and a diameter D3b may represent a diameter of the second subset 180b and the second subset 182b formed by struts of the respective first and second end sections 112a, 112b. The diameters D3a, D3b may be substantially larger than diameter D2a or D2b. In some embodiments, diameter D3a and diameter D3b may be about 5 percent to about 30 percent larger than diameter D2a or D2b; about 8 percent to about 15 percent larger than diameter D2a or D2b; or about 15 percent to about 25 percent larger than diameter D2a or D2b. In some embodiments, diameter D3a and diameter D3b may be about 14 percent larger than diameter D2a or D2b

A diameter D4a and a diameter D4b may represent a diameter of the third subset 180c and the third subset 182c formed by struts of the respective first and second end sections 112a, 112b. The diameters D4a, D4b may be substantially larger than to diameter D3a or D3b. In some embodiments, diameter D4a and diameter D4b may be about 8 percent to about 20 percent larger than diameter D3a or D3b; about 8 percent to about 15 percent larger than diameter D3a or D3b; or about 15 percent to about 20 percent larger than diameter D3a or D3b. In some embodiments, diameter D4a and diameter D4b may be about 12 percent larger than diameter D3a or D3b

The stents described herein may vary strut length over at least portions of the length of the stent, as described above, to avoid a problem that occurs with conventional stents that have strut lengths of the same length over the length of the stent. For example, conventional stents with conventionally similar strut lengths can be problematic in small, tortuous anatomy because as the stent diameter is increased, the angles at which the struts engage with vessel walls change. In particular, as the stent diameter increases, the angle at which the struts engage with vessel walls increases causing instability of the stent itself and/or instability related to anchoring the stent. For example, for a strut length of about 1.5 mm, the diameter may be increased. Table 1 below indicates an example of crown angle measurements as the stent diameter is increased.

TABLE 1

| | Stent end section |
|---|---|
| Stent Diameter (mm) | Crown Angle (degrees) |
| 5 | 80.54 |
| 5.5 | 90.64 |
| 6 | 101.74 |
| 7 | 129 |

As the stent diameter increases, the angle of the struts increases. For example, with a stent diameter of about 5 mm, the angle of the struts on particular end sections of the stent reaches about 80.5 degrees. As the stent diameter is raised to about 5.5 mm, the angle of the struts on the end sections of the stent increases to about 90.6 degrees. Similarly, an increase to 6 mm in stent diameter results in a crown angle increase up to about 101.7 degrees and an increase to about 7.0 mm results in a crown angle increase up to about 129 degrees. In general, crown angles over 90 degrees cause instability of the stent. Maintaining the crown angle for the end portions of the stent to be about 80 degrees or lower can allow stable anchoring to a vessel wall and/or reduce malapposition between the stent and the vessel wall. Such stability allows anchoring without inward or outward strut bending, which can cause blocking of the lumen.

In some embodiments, the stent 100 may exhibit particular behavior responsive to radial compressive forces imposed on the stent as the stent supports walls of a vessel. For example, the stent 100 may have a radial resistive force (i.e., radial strength, radial force) based on the structure and assembly of the stent. In particular, the length, thickness, and/or flexion of the struts of the stent may be used to vary or maintain the radial force along the length of the stent. For example, increasing the strut length for struts that begin at an end portion of the body section 114 and continuing to increase struts along the end portions (e.g., end section 112a, end section 112b) can attenuate how much a crown angle is able to be increased in the end section 112a, 112b (i.e., flared portions) of the stent 100. Thus, the stents described herein may progressively increase the strut lengths in the end sections 112a, 112b to maintain a crown angle of less than about 80 degrees and to maintain a substantially constant radial force along the length of the stent, each of which can ensure stability of the stent within the vessel. In addition, an angle at which particular struts are positioned may also be used to vary the radial force along the length of the stent. Varying the radial force may include changing one or more of: strut length, crown angle configuration, strut material, strut thickness, and/or strut flexion to ensure that the radial force remains relatively constant along the length of the stent during deployment, expansion, and/or compression. In some embodiments, particular lengths of struts are used in particular locations along the stent to ensure that such radial forces may remain relatively stable within a predefined range, as described in detail herein.

In some embodiments, the body section 114 that includes the third plurality of struts (e.g., struts 170 of FIG. 1B) may have a first radial force at a first end (shown by diameter D1a in FIG. 1C) of the body section 114 and may have substantially the same radial force at a second end (shown by diameter D1b in FIG. 1C) of the body section 114. The first plurality of struts (e.g., subset 120a, subset 120b, and subset 120c) of the first end section 112a of the stent 100 may be arranged to have a second radial force that substantially matches the first radial force. For example, the first radial force and the second radial force may be between about 0.4 N/mm to about 0.5 N/mm when the stent 100 is at about 2 mm of compression. The second plurality of struts (e.g., subsets of struts 120d, 120e, and 120f) of the second end section 112b of stent 100 may be arranged to have a third radial force that substantially matches the first radial force. For example, the first radial force and the third radial force may be between about 0.4 N/mm to about 0.5 N/mm when the stent 100 is at about 2 mm of compression. FIG. 2 shows the shape-set configuration of the stent of FIGS. 1A-1B, with a substantially constant diameter D of the body section 114, and rings increasing in diameter in the first end section 112a and the second end section 112b, moving from the body section 114 towards the proximal and distal faces 102, 104, respectively. For example, the diameter of the first end section 112a may variably increase in size from the substantially cylindrical body section 114 toward the first end section 112a (e.g., end 102) by about 40 percent to about 80 percent; about 40 percent to about 50 percent; about 50 percent to about 60 percent; about 60 percent to about 70 percent; or about 70 percent to about 80 percent.

In addition, the diameter of the second end section 112b may variably increase in size from the substantially cylindrical body section 114 toward the second end section 112a (e.g., end 104) by about 40 percent to about 80 percent; about 40 percent to about 50 percent; about 50 percent to about 60 percent; about 60 percent to about 70 percent; or about 70 percent to about 80 percent.

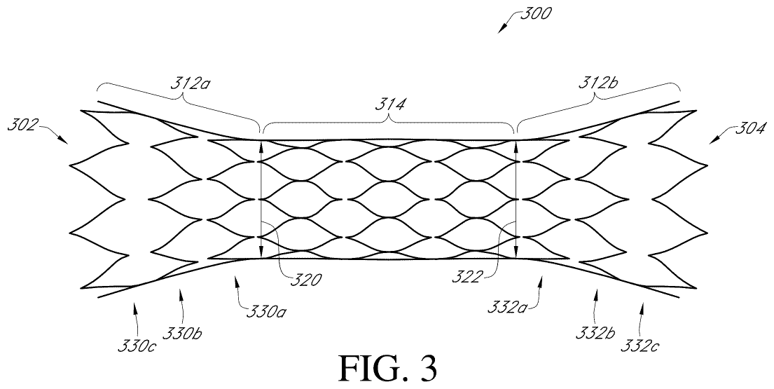

The stent 300 of FIG. 3 is similar to that shown in FIGS. 1A-2, except in this embodiment, the body section 314 is longer than the body section 114 shown in FIGS. 1A-2. For example, body section 314 of stent 300 includes 4 rings to 10 rings and the first and second end sections 312a, 312b include three rings each. As above with respect to FIGS. 1A-2, the struts length increases in the first and second end sections (terminal struts are longer than penultimate struts, which are longer than antepenultimate struts), moving from the body section 314 towards the proximal face 302 or distal face 304, respectively.

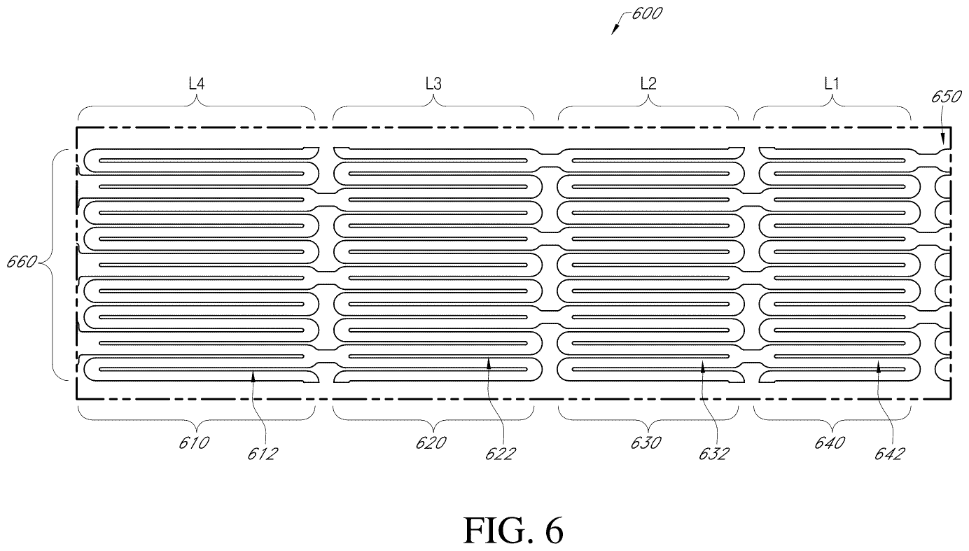

In some embodiments, the body section 314 includes a third plurality of struts (e.g., 4 rings to 10 rings that include struts of the body section 114) may have a first radial force at a first end 320 of the body section 314 and may have substantially the same radial force at a second end 322 of the body section 314. The end section 312a may include a ring 330a, a ring 330b, and a ring 330c, which may be similar to rings 610, 620, 630, 640, etc. of FIG. 6. The rings 330a, 330b, 330c may have a first plurality of struts arranged to exert a second radial force that substantially matches the first radial force. For example, the first radial force and the second radial force of stent 300 may be between about 0.4 N/mm to about 0.5 N/mm when the stent 300 is at about 2 mm of compression.

Similarly, the end section 312b may include a ring 332a, a ring 332b, and a ring 332c—which may be similar to rings 610, 620, 630, 640, etc. of FIG. 6. The rings 332a, 332b, 332c may have a first plurality of struts arranged to exert a third radial force that substantially matches the first radial force. For example, the first radial force and the third radial force may be between about 0.4 N/mm to about 0.5 N/mm when the stent 100 is at about 2 mm of compression.

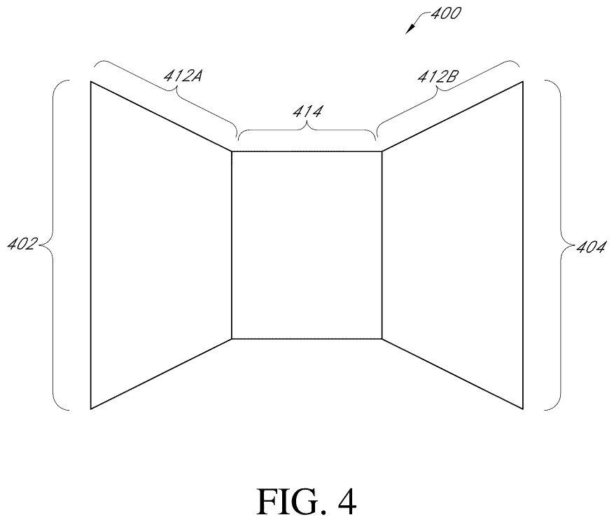

The stent 400 of FIG. 4 is similar to that shown in FIGS. 1A-2, except in this embodiment, the body section 414 is shorter than the body section 114 shown in FIGS. 1A-2. For example, body section 414 includes 1 ring to 4 rings or about 3 rings, resulting in a shorter body section 414. As above with respect to FIGS. 1A-2, the struts length increases in the first and second end sections 412a, 412b (terminal struts are longer than penultimate struts, which are longer than antepenultimate struts), moving from the body section 414 towards the proximal face 402 or distal face 404, respectively,

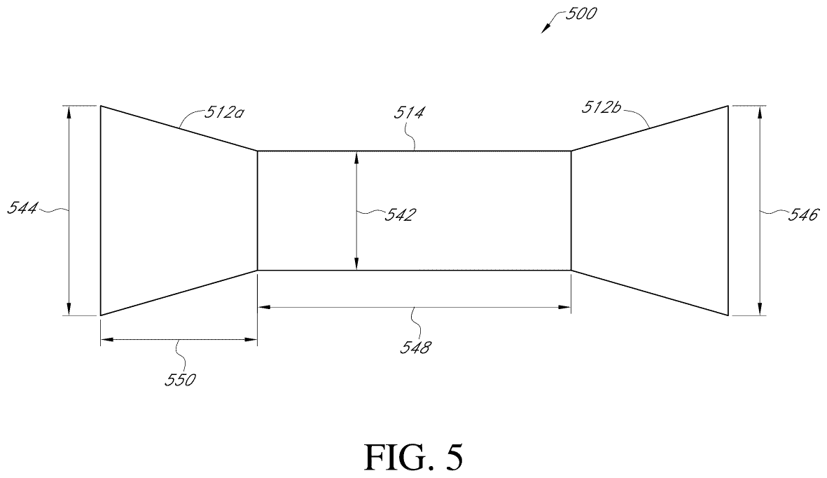

FIG. 5 shows one example of various parameters of the stent 500, at least for a particular vessel having particular dimensions. One of skill in the art will appreciate that the dimensions will generally be scaled up or down depending on the size of the target vessel. Stent 500, in an expanded configuration, has a body section 514 having a length 548 of about 6 mm to about 12 mm, preferably about 8 mm to about 10 mm, depending on a target vessel length specification. Stent 500 has a first end section 512a and a second end section 512b, each having a length 550 of about 3.0 mm to about 6 mm, preferably between about 4.0 mm to about 5 mm. A diameter 542 of the body section 314 may be about 3.0 mm to about 6.0 mm, preferably about 3.5 mm to about 4.5 mm, depending on target vessel specification. A diameter 544 of the proximal face or a diameter 536 of the distal face may be about 6.0 mm to about 8.0 mm, preferably about 6.5 mm to about 7.5 mm, depending on target vessel specifications.

FIG. 6 shows an end section 600 including a plurality of rings 610, 620, 630, 640, each including a plurality of struts 612, 622, 632, 642, respectively. Each of the plurality of struts in each ring has a length. For example, each of the plurality of struts 612 including terminal ring 610 has a length L4; each of the plurality of struts 622 including penultimate ring 620 has a length L3; each of the plurality of struts 632 including antepenultimate ring 630 has a length L2; and each of the plurality of struts 642 including fourth ring 640 has a length L1. In this embodiment, the strut length increases on a row by row basis, for example incrementally, moving from the body section 650 of the stent towards the distal or proximal faces 660. The strut length increases on a row by row basis by about 10 percent to about 30 percent or by about 12 percent to about 25 percent. As shown in FIG. 6, length L1 to length L2 may increase by about 15 percent to about 25 percent, preferably about 20 percent; length L2 to length L3 may increase by about 10 percent to about 15 percent, preferably about 13.3 percent, and length L3 to length L4 may increase by about 10 percent to about 20 percent, preferably about 14.7 percent. In some embodiments, L4 may have a length of about 1.7 mm to about 2.0 mm; about 1.8 mm to about 1.9 mm; or about 1.9 mm. In some embodiments, L3 may have a length of about 1.5 mm to about 1.9 mm. about 1.6 mm to about 1.8 mm; about 1.6 mm to about 1.7 mm; or about 1.7 mm. In some embodiments, the length L2 may have a length of about 1.4 mm to about 1.6 mm; about 1.4 mm to about 1.5 mm; about 1.5 to about 1.6 mm; or about 1.55 mm. In some embodiments, the length L1 may have a length of about 1.4 mm to about 1.6 mm; about 1.4 mm to about 1.5 mm; about 1.5 to about 1.6 mm; or about 1.53 mm.

In some embodiments, the axial lengths of the rings when in the expanded state may include a main body L1 at a length of about 1.55 mm; L2 at about 1.6 mm; L3 at about 1.65 mm; and L4 at about 1.69 mm.

FIG. 7 shows the dimensions of the crown angles remaining substantially constant as the stent is expanded to the target diameters. For example, adjacent struts 712a and 712b; 722a and 722b; 732a and 732b; or 742a and 742b in a ring 710, 720, 730, 740, respectively, may form an angle A1, A2, A3, A4, respectively, of about 50 degrees to about 70 degrees, preferably about 65 degrees, to maximize a radial force exerted by the stent on the vessel. In some embodiments, to maintain a substantially constant angle between the struts, the length of the struts increases in each ring, moving from a body section towards a distal or proximal face, as shown above in FIG. 6.

Alternatively, the crown angles may change as the stent is expanded to the target diameters. For example, A3 may be larger than A4, A2 may be larger than A3 and A4, and A1 may be larger than A2, A3, and A4. In some embodiments, the angle of A1 may be about 55 degrees to about 60 degrees; about 55 degrees to about 58 degrees; about 55 degrees to about 57 degrees; about 56 degrees to about 58 degrees; or about 57 degrees to about 58 degrees. In some embodiments, the angle of A2 may be about 55 degrees to about 65 degrees; about 55 degrees to about 63 degrees; about 59 degrees to about 61 degrees; or about 59 degrees to about 60 degrees. In some embodiments, the angle of A3 may be about 60 degrees to about 70 degrees; about 60 degrees to about 65 degrees; about 65 degrees to about 68 degrees; about 65 degrees to about 67 degrees; or about 66 degrees to about 67 degrees. In some embodiments, the angle of A4 may be about 65 degrees to about 75 degrees; about 65 degrees to about 72 degrees; about 65 degrees to about 70 degrees; about 68 degrees to about 70 degrees; or about 69 degrees to about 70 degrees.

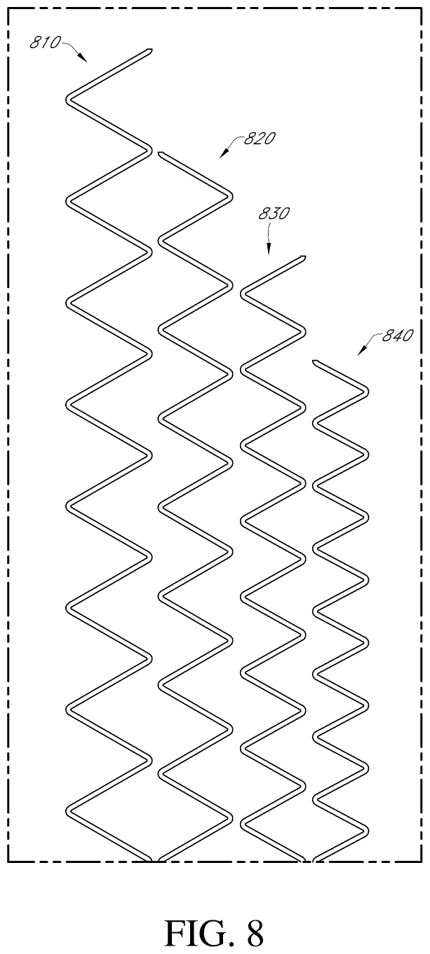

FIG. 8 shows example diameters (in a two-dimensional representation) of each ring 810, 820, 830, 840 as the stent is expanded. Terminal ring 810 has a diameter (when in a three-dimensional shape) of about 6 mm to about 8 mm; the penultimate ring 820 has a diameter (when in a three-dimensional shape) of about 5 mm to about 7 mm; the antepenultimate ring 830 has a diameter (when in a three-dimensional shape) of about 4 mm to about 6 mm; and the fourth from the end ring 840 has a diameter (when in a three-dimensional shape) of about 3 mm to about 5 mm. As such, the stent outer diameter increases by about 40 percent to about 80 percent from the body section to the distal or proximal faces.

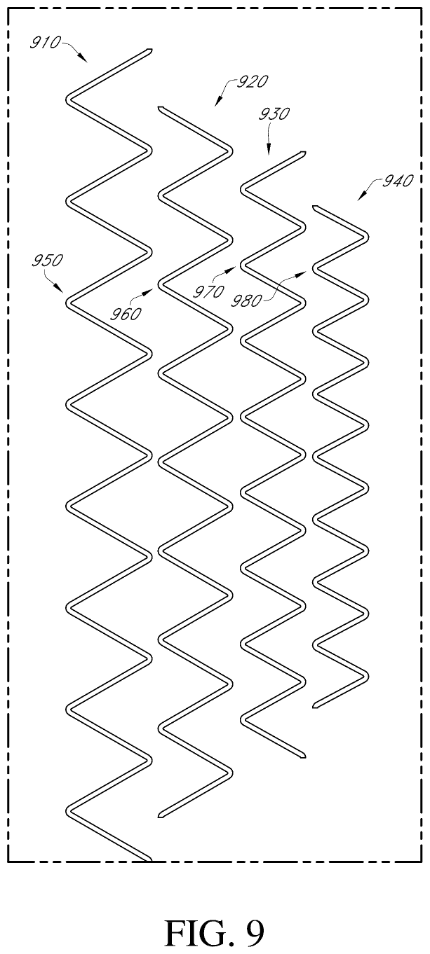

FIG. 9 shows the ring alignment between adjacent rings, for example terminal ring 910 and penultimate ring 920; penultimate ring 920 and antepenultimate ring 930; and antepenultimate ring 930 and fourth ring 940, when expanded into the flared shape. For example, the crowns are aligned or in phase as shown in FIG. 9. For example, terminal crown 950 is aligned with penultimate crown 960 which is aligned with antepenultimate crown 970 which is aligned with fourth crown 980. The bridges (not shown) may be positioned and/or may maintain the crowns in adjacent rings in alignment.

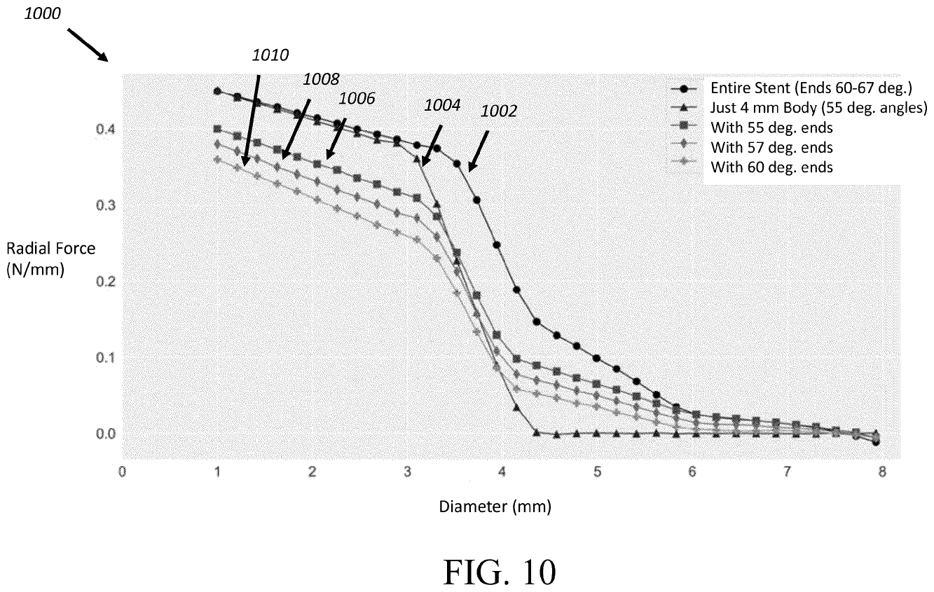

FIG. 10 shows a graphical representation 1000 of example radial forces versus diameter for the stents described herein. The graphical representation 1000 depicts how a changing crown angle (similar to Table 1 above) can rapidly devolve into an unusable stent because the radial resistive force begins to decrease and then fail as the stent diameter is increased when the strut size is designed using the same strut length size over the entire stent length.

The data shown in graphical representation 1000 was obtained from a benchtop test to offer empirical evidence of the radial force exerted by a self-expanding stent (such as the stents described herein) as a function of the stent diameter under the conditions of expansion and compression. The samples were deployed within a testing fixture iris until the initial diameter approached less than or equal to a minimum vessel diameter. Typically, such radial force testing involves compressing cylindrical stents and measuring the diameter change and hoop force. Test equipment measures and records device characteristics including radial stiffness and strength, chronic outward force during expansion and radial reactive force during compression. The equipment has the capability to display force outputs in various units of hoop force, radial force or pressure. Radial force is the plotted as the force per unit length of the stent, versus the diameter of the stent.

As shown in FIG. 10, a first line 1002 represents the radial force of an entire stent with angles between adjacent struts at the ends between about 60 degrees and about 67 degrees. A second line 1004 represents a 4 mm body portion of the stent with an angle between adjacent struts of the body being about 55 degrees. A third line 1006 represents the 4 mm body portion of the stent with the ends of the stent having angles between adjacent struts of about 55 degrees. A fourth line 1008 represents the 4 mm body portion of the stent with the ends of the stent having angles between adjacent struts of about 57 degrees. A fifth line 1010 represents the 4 mm body portion of the stent with ends of the stent having angles between adjacent struts of about 60 degrees. The radial force performs well between about a 3 mm stent and a 4.5 mm stent. For example, when the stent is compressed from about 4.5 mm to about 3 mm, the stents described herein with the crown angles below about 80 percent and the stent lengths being progressively increased from the body section 114 toward end sections 112a, 112b, the radial force can be maintained at a substantially constant level across the length of the stent 100. Lower crown angles on the ends of the stent (caused by progressively increasing strut lengths moving away from the stent body) allows for maintaining or increasing radial force at the ends to improve and/or maintain stent anchoring and stability. The data of graphical representation 1000 teaches away from using constant strut lengths over the entire stent length as using constant strut lengths can cause stent instability as stent diameter is increased.

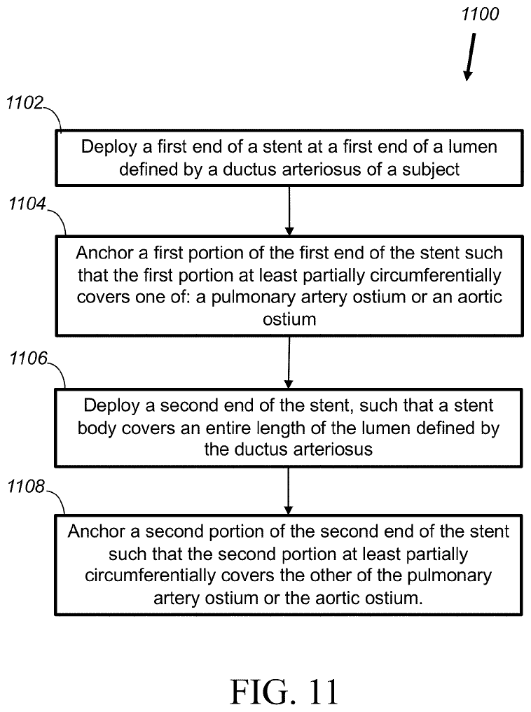

FIG. 11 shows a flow diagram of an example method of treatment 1100 of ductus arteriosus using at least one of the stents described herein. The method of treatment 1100 may be described with respect to a particular stent 100, for example, however, one skilled in the art will appreciate that any of the stents described herein (e.g., stent 300, stent 400, stent 500) may be substituted for stent 100 when performing the method of treatment 1100. In some embodiments, the stents described herein may be used to provide treatment of a ductus arteriosus in a pediatric patient. For example, the stents described herein may be used in the method of treatment 1100 to maintain a patent ductus arteriosus in a pediatric patient to increase a pulmonary circulation of the pediatric patient. In some embodiments, the stent used in method 1100 may include a self-expanding stent. In some embodiments, the stent used in the method of treatment 1100 may be a user expandable stent.

At block 1102, the method of treatment 1100 may include deploying a first end (e.g., proximal face 102/end section 112a) of a stent at a first end of a lumen defined by a ductus arteriosus of a subject. For example, stent 100 may be introduced (e.g., inserted) to and deployed within a vessel (e.g., ductus arteriosus) or tissue site using a delivery system, as described above.

At block 1104, the method of treatment 1100 may include anchoring a first portion of a first end of the stent such that the first portion at least partially circumferentially covers one of: a pulmonary artery ostium or an aortic ostium. For example, the first portion may include one or more flanges, rings, loops, corners, or other stent portion that may anchor the first end (e.g., some or all of proximal face 102/end section 112a) of the stent 100 to interact (e.g., contact, compress, friction fit, etc.) with the walls of the vessel in which the stent 100 is installed. In addition, one or more of a stiffness, size, longitudinal angle, or circumferential angle of an end portion (e.g., some or all of either end section 112a and/or end section 112b) of stent 100 are adjustable by altering strut thickness, length, and/or heat shaping parameters to tune the level of engagement of the first end portion of the stent 100 with the vessel wall and amount of force they exert on the vessel wall. In some embodiments, the strut thickness remains constant across the struts, while the strut length is progressively increased for portions of the end sections 112a, 112b beginning from the body section 114 and moving toward one or both end sections 112a, 112b.

At block 1106, the method of treatment 1100 may include deploying a second end of the stent. For example, stent 100 may be introduced to and deployed within a vessel (e.g., ductus arteriosus) or tissue site using a delivery system, as described above. In some embodiments, the stent 100 may be deployed until a stent body covers (e.g., substantially covers or substantially lines or substantially runs) an entire length of the lumen defined by the ductus arteriosus. In some embodiments, the stent 100 may be deployed until a stent body substantially covers between about 98 percent and about 100 percent of a length of the lumen defined by the ductus arteriosus.

At block 1108, the method of treatment 1100 may include anchoring a second portion of the second end of the stent such that the second portion at least partially circumferentially covers the other of the pulmonary artery ostium or the aortic ostium. For example, the second portion may include one or more flanges, rings, loops, corners, or other stent portion that may anchor the second end (e.g., some or all of end section 112b) of the stent 100 to interact (e.g., contact, compress, friction fit, etc.) with the walls of the vessel in which the stent 100 is installed. In addition, one or more of a stiffness, size, longitudinal angle, or circumferential angle of an end portion (e.g., some or all of end section 112a and/or end section 112b) of stent 100 are adjustable by altering strut thickness, length, and/or heat shaping parameters to tune the level of engagement of the second end portion of the stent 100 with the vessel wall and amount of force they exert on the vessel wall.

In general, the anchoring of the first portion of the first end (e.g., some or all of end section 112a) of the stent 100 and the anchoring of the second portion of the second end (e.g., some or all of end section 112b) of the stent 100 may function to maintain a patent ductus arteriosus for the subject (e.g., a pediatric patient). The stent may remain deployed and anchored for a period of time to support the surrounding tissue of the ductus arteriosus and ensure patency for at least about one month or longer when the patient no longer receives prostaglandins, for example.

In some embodiments, the stents described herein are made using Nitinol®, which is self-expanding and can be tailored to have a sufficient radial force to maintain a patent ductus lumen. This radial force can be precisely dialed in during the design process and then maintained in manufacturing. Further, it will be appreciated that any of the stents described herein may be made of or comprise a self-expanding shape memory alloy, such as, for example, copper-aluminum-nickel, nickel-titanium (i.e., Nitinol®), iron-manganese-silicon, or copper-zinc-aluminum. The flexibility and stretchability of the material is conducive to ensuring access to and through a patient's tortuous anatomy while also having a radial outward force sufficient enough to support the surrounding tissue of the ductus arteriosus to ensure patency for at least about one month or longer when the patient no longer receives prostaglandins, for example.

For any of the stent embodiments described herein, stent flexibility may be increased by reducing connecting member width, for example. Additionally, or alternatively, any of the stent embodiments described herein may be treated with regioselective heat and/or regioselective wall thickness removal (either by selective bead-blasting or masked chem-etching) to increase flexibility of the stent. Additionally, or alternatively, one or more of a stiffness, size, longitudinal angle, or circumferential angle of the anchoring ends are adjustable by altering a strut thickness, length, and/or heat shaping parameters as well as other levers to tune the level of engagement of the anchoring portions of the stent with the vessel wall and amount of force they exert on the vessel walls. Further, any of the stent embodiments described herein may be heat shaped to create varying curves and angles without performing alterations to the laser cut patterns.

In some embodiments, any of the stents described herein may comprise any one or more of the following coatings or sleeves on an inner diameter, outer diameter, or along an entire length of the device (inner and outer diameter): an anti-thrombogenic, anti-restenotic, lubricious, etc.

Although the figures shown herein include both the proximal and distal ends of the stent as having flared ends, one of skill in the art will appreciate that one end may be a flared end while the other end may not be flared, and therefore may not be considered to be anchoring. In some embodiments, one or both flared ends may prevent or reduce migration of the stent during insertion, deployment and/or while positioned in the patient for an extended period of time, as described elsewhere herein.

As used herein, the term “user” may include, but should not be limited to, a physician, assistant, doctor, nurse, interventionalist, healthcare provider, technician, radiologist, or the like.

As used herein, the terms “patient” or “subject” may include, but not be limited to, a fetus, neonate, pediatric, toddler, pre-mature baby, baby, or the like.

As used herein, the terms “ductus” and “ductus arteriosus” may be used interchangeably.

In some embodiments, as used herein, the phrase “an entire length of the ductus” may be measured from aorta ostium to pulmonary artery ostium, based on anatomical imaging, measured from a first ductal end (e.g., at the aorta) to a second ductal end (e.g., at the pulmonary artery), measured along the outer edge of the ductus curvature, measured along the inner edge of the ductus curvature, measured through the centerline of the ductus curvature, or the like.

As used herein, the terms “proximal” and “distal” depend on the approach taken with a delivery system. For example, if approaching the ductus from the aorta, then the pulmonary artery may be considered to be distal with respect to the aorta and delivery system. If approaching the ductus from the pulmonary artery, then the aorta may be considered to be distal with respect to the pulmonary artery and delivery system. As such, in some cases, first and second ends are used to replace proximal and distal terminology to illustrate the interchangeability of these terms and their dependency on the type of procedure being performed.

As used in the description and claims, the singular form “a”, “an” and “the” include both singular and plural references unless the context clearly dictates otherwise. For example, the term “struts” may include, and is contemplated to include, a plurality of struts. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5 percent, 1 percent or 0.1 percent. All numerical ranges provided herein are inclusive of the stated start and end numbers. The term “substantially” indicates mostly (i.e., greater than 50 percent) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A device for insertion into a blood vessel lumen to provide a generally radially outward force on the blood vessel lumen, comprising:
a first end section comprising a first plurality of struts having a first diameter and defining a proximal face, wherein a length of the first plurality of struts progressively increases towards the proximal face, and wherein a first crown angle between adjacent struts of the first plurality of struts progressively increases towards the proximal face;
a second end section comprising a second plurality of struts having a second diameter and defining a distal face, wherein a length of the second plurality of struts progressively increases towards the distal face, and wherein a second crown angle between adjacent struts of the second plurality of struts progressively increases towards the distal face;
a body section extending between the first end section and the second end section and comprising a third plurality of struts; and
a device lumen extending through the first end section, the body section, and the second end section, the device lumen being configured to allow blood flow through the device, wherein, in an expanded configuration:
the first diameter of the first end section increases from the body section towards the proximal face, and the second diameter of the second end section increases from the body section towards the distal face, and
a radial force of the device is substantially constant from the first end section to the body section to the second end section when each section is at substantially a same diameter.

2. The device of claim 1, wherein the device is configured for maintaining a patent ductus arteriosus.

3. (canceled)

4. The device of claim 1, wherein the radial force is between about 0.4 N/mm to about 0.5 N/mm when the device is at about 2 mm of compression.

5. The device of claim 1, wherein the radial force is between about 0.2 N/mm to about 0.6 N/mm when the device is at about 2 mm of compression.

6. The device of claim 1, wherein a length of a first subset of the first plurality of struts is increased by about 5 percent to about 25 percent moving from the body section towards the proximal face; and a length of a first subset of the second plurality of struts is increased by about 12 percent to about 25 percent moving from the body section towards the distal face.

7. (canceled)

8. The device of claim 1, wherein each of the third plurality of struts is substantially equal in length.

9. The device of claim 6, wherein:
the first subset of the first plurality of struts have the length of about 1 mm to about 2 mm; and
the first subset of the second plurality of struts have the length of about 1 mm to about 2.1 mm.

10. The device of claim 6, further comprising a second subset of struts in the first plurality of struts and a second subset of struts in the second plurality of struts, wherein:
the second subset of struts in the first plurality of struts have a length of about 1.2 mm to about 2.5 mm; and
the second subset of struts in the second plurality of struts have a length of about 1.4 mm to about 2.5 mm.

11. The device of claim 10, further comprising a third subset of struts in the first plurality of struts and a third subset of struts in the second plurality of struts, wherein:
the third subset of struts in the first plurality of struts have a length of about 1.9 mm to about 2.0 mm; and
the third subset of struts in the second plurality of struts have a length of about 1.9 mm to about 2.0 mm.

12. The device of claim 1, wherein the first diameter of the first end section is increased from the body section towards the proximal face by about 40 percent to about 80 percent, such that the first end section has a flare shape that flares from the body section toward the proximal face.

13. (canceled)

14. The device of claim 1, wherein the second diameter of the second end section is increased from the body section towards the distal face by about 40 percent to about 80 percent, such that the second end section has a flare shape that flares from the body section toward the distal face.

15. (canceled)

16. The device of claim 1, wherein the first plurality of struts and the second plurality of struts are arranged in rings.

17. The device of claim 16, wherein there are between about 3 rings to about 5 rings comprising each of the first end section and the second end section.

18. The device of claim 16, wherein the progressive increase in first and second diameters moving from the body section either proximally or distally, respectively, is incremental on a ring-by-ring basis.

19. The device of claim 18, wherein the increment is an increase in the first and second diameters of between about 10 percent to about 30 percent.

20. The device of claim 16, wherein the radial force ranges from about 0.1 N/mm to about 0.4 N/mm at 1 mm of compression to about 0.1 N/mm to about 0.6 N/mm at 2 mm of compression.

21. The device of claim 16, wherein each ring is connected to an adjacent ring by about 3 bridges to about 9 bridges, and wherein the bridges are positioned such that crowns in adjacent rings are substantially aligned.

22. (canceled)

23. The device of claim 1, wherein one or both of: the proximal face and the distal face has a diameter that is about 10 percent to about 80 percent larger than a diameter of the body section.

24. (canceled)

25. The device of claim 1, wherein the device is configured to be positioned in a bodily lumen comprising a procedure diameter that is larger than a post-procedure diameter, and wherein the first and second end sections are configured to anchor the device in the bodily lumen having the procedure diameter.

26. The device of claim 25, wherein the procedure diameter of the bodily lumen is a result of prostaglandin administration to a patient.

27.-29. (canceled)
